(19) United States
(12) Patent Application Publication
(10) Pub. No.: US 2024/0052291 A1
(43) Pub. Date: Feb. 15, 2024

(54) A BIOPROCESSING SYSTEM AND METHOD

(71) Applicant: CELLULAREVOLUTION LTD, Tyne and Wear (GB)

(72) Inventors: Che CONNON, Tyne and Wear (GB); Caroline BELLANI, Tyne and Wear (GB)

(21) Appl. No.: 18/267,474

(22) PCT Filed: Dec. 1, 2021
(86) PCT No.: PCT/GB2021/053135

§ 371 Date: Jun. 15, 2023

(30) Foreign Application Priority Data
Dec. 15, 2020 (GB) 2019793.5

(51) Int. Cl. C12M 1/00; C12M 1/26; C12M 1/36; C12M 1/34

(52) U.S. Cl. CPC C12M 47/04; C12M 33/14; C12M 29/18; C12M 41/48; C12M 41/36

(57) ABSTRACT

The present invention relates to a system for continuous bioprocessing of adherent cells. The system includes a cell growth chamber; a cell trap in fluid communication with the cell growth chamber, the cell trap including a cell outlet; and a fluid flow system configured to selectively switch between a first mode of operation and a second mode of operation. In the first mode of operation the fluid flow system is configured to direct fluid through the cell growth chamber towards the cell trap such that cells from the cell growth chamber are collected in the cell trap. In the second mode of operation the fluid flow system is configured to direct fluid to the cell trap and through the cell outlet to thereby harvest cells collected in the cell trap.

The present invention relates to a system for continuous bioprocessing of adherent cells, and use thereof. The invention also relates to a method for continuous bioprocessing of adherent cells.

### BACKGROUND

It is well-known that regenerative medicine, and in particular cell-therapy techniques, has been expanding its repertoire in terms of both purposeful cell types and applications, raising the potential to cure a wide range of diseases. However, the high number of cells required for some treatments (this can be up to 1 billion) presents, in terms of cell manufacture, a major challenge.

Furthermore, as the final product is often represented by the cells themselves, numerous requirements have to be met under the current regulatory framework for cellular therapy products. In particular, the system used for cell expansion has to be compliant with the current good manufacturing practices; the fluid path should be closed and sterile while minimizing human intervention; the culture media should be chemically defined and preferably not contain serum or other xenobiotics; and the process should be standardized, reproducible, and in general possessing characteristics that maintain the desired cell phenotype throughout the entire process while ensuring reasonable costs of goods are maintained.

Thus, implementing and adapting industrial processes, previously developed for large-scale mammalian cell culture for the production of biologics, has been attempted in order to sustain such a great demand for cells and to meet the required conditions.

Of these processes, one of the most advanced methods for obtaining large numbers of adherent cells is through automation or microcarrier-based expansion using perfusion bioreactors. While substantial increases in cell yield have been achieved by improving microcarrier function, some other critical production steps are still undergoing optimization. For instance, improved cell harvesting methods for easier, more effective and less invasive recovery of the expanded cells from their microcarrier culture substrates are required. For these reasons and others such as cost, microcarrier preparation, and the time/resources required to translate established 2D flask-based protocols to 3D microcarrier cultures, the uptake of microcarrier-based systems by commercial organizations has been slow.

A continuous bioprocessing system could meet all the required criteria for therapeutic cell expansion, in terms of both efficiency and regulatory compliance.

The general concept of continuous bioprocessing, introduced more than 30 years ago, denotes a closed system that operates unremittingly, processing the constantly flowing raw material into the intermediate or final products. There are several advantages to such a system, including reduction in manufacturing costs and in the size of manufacturing facilities, as well as the improvements in quality, reproducibility, and standardization of the final product.

Continuous bioprocessing is currently being applied in biotechnology (e.g., for the production of biopharmaceuticals, recombinant proteins, and monoclonal antibodies).

However, this methodology is not currently being implemented for the manufacture of cells with therapeutic potential due to a lack of suitable continuous bioprocessing methods.

Accordingly, it is an object of the invention to provide a system for continuous bioprocessing of adherent cells that would allow culturing and harvesting of adherent cells with minimal human intervention in a system for continuous bioprocessing of cells, and thereby ameliorate some of the problems associated with the prior art.

### SUMMARY OF THE INVENTION

According to a first aspect of the invention, there is provided a system for continuous bioprocessing of adherent cells, the system comprising:
a cell growth chamber;
a cell trap in fluid communication with cell growth chamber, the cell trap including a cell outlet; and
a fluid flow system configured to selectively switch between a first mode of operation and a second mode of operation;
wherein, in the first mode of operation the fluid flow system is configured to direct fluid through the cell growth chamber towards the cell trap such that cells from the cell growth chamber are collected in the cell trap, and
in the second mode of operation the fluid flow system is configured to direct fluid to the cell trap and through the cell outlet to thereby harvest cells collected in the cell trap.

The cell growth chamber of the system may include any of the features of the cell growth chamber described herein.

Suitably, the system further comprises at least one reservoir for introducing fluids into the fluid flow system.

Suitably, in the first mode of operation the fluid flow system is configured to direct fluid from the cell trap towards a fluid waste outlet or back through the cell growth chamber.

Suitably, the cell trap of the system comprises a filter configured to prevent passage of cells towards the fluid waste outlet or back through the cell growth chamber.

Suitably, in the second mode of operation the fluid flow system is configured such that fluid by-passes the cell growth chamber.

Suitably, the system further comprises a valve assembly configured to selectively switch between a first configuration corresponding to the first mode of operation and a second configuration corresponding to the second mode of operation.

Suitably, in the first configuration the valve assembly is configured to prevent flow of fluid directly to the cell trap without first passing through the cell growth chamber.

Suitably, in the first configuration the valve assembly is configured to allow flow of fluid from a fluid reservoir to the cell growth chamber.

Suitably, in the first configuration the valve assembly is configured to allow passage of medium along the growth chamber inlet conduit and prevent passage of medium along the by-pass conduit.

Suitably, in the second configuration the valve assembly is configured to prevent flow of fluid from a fluid reservoir to the cell growth chamber.

Suitably, in the second configuration the valve assembly is configured to allow flow of fluid from a fluid reservoir directly to the cell trap.

Suitably, in the second configuration the valve assembly is configured to prevent passage of medium along the growth chamber inlet conduit and prevent passage of medium along the by-pass conduit.

Suitably, the valve assembly comprises a first valve configured to selectively control flow of fluid to the cell growth chamber and the cell trap, and a second valve configured to selectively allow flow of fluid through the cell outlet.

Suitably, the system further comprises a by-pass conduit configured for passage of fluid to the cell trap from a fluid reservoir.

Suitably, the system further comprises a growth chamber inlet conduit configured for passage of fluid from a fluid reservoir to the growth chamber.

Suitably, the system further comprises at least one pump for pumping fluid through the fluid flow system.

Suitably, the at least one pump is not positioned between the cell growth chamber and the cell trap.

Suitably, the system further comprises a collection container fluidly coupled to the cell trap for collecting harvested cells from the cell trap.

Suitably, the collection container is detachable from the system.

Suitably, the cell growth chamber of the system comprises an inlet, an outlet, and one or a plurality of cell culture plates positioned between the inlet and the outlet.

Suitably, the plurality of cell culture plates are arranged in a stacked configuration defining a plurality of flow channels through the cell growth chamber, and are oriented such that, in use, cells detached from the cell culture plates travel along the flow channels and through the outlet.

Suitably, the system further comprises at least one sensor configured to monitor at least one fluid property of fluid in the system.

Suitably, the system further comprises a sensor configured to monitor the volume of cells within the cell trap.

Suitably, the system further comprises a controller configured to automatically control the fluid flow system to switch between the first mode of operation and the second mode of operation.

Suitably, the controller is configured to automatically switch the fluid flow system to the second mode of operation when a quantity of cells collected in the cell trap reaches a predetermined threshold.

According to a second aspect of the invention, provided herein is use of the system according to any preceding claim for continuous bioprocessing of adherent cells.

According to a third aspect of the invention, provided herein is a method for continuous bioprocessing of adherent cells, the method comprising:
selectively switching a fluid flow system between a first mode of operation and a second mode of operation,
wherein the first mode of operation comprises directing fluid through a cell growth chamber towards a cell trap and collecting cells from the cell growth chamber in the cell trap,
wherein the second mode of operation comprises directing fluid to the cell trap and through a cell trap outlet to thereby harvest cells collected in the cell trap.

It will be appreciated that the method above may be carried out using a system including any of the features described above or discussed herein.

Suitably, in the first mode of operation, fluid exiting the cell trap is recirculated through the cell growth chamber.

Suitably, in the first mode of operation, at least a portion of fluid exiting the cell trap is selectively directed towards a waste circuit and removed from the fluid flow system.

Described herein is also a cell growth chamber for continuous bioprocessing of adherent cells, the cell growth chamber comprising:
an inlet positioned at an upper end of the cell growth chamber, wherein the inlet is couplable to a cell culture medium source;
an outlet positioned at a lower end of the cell growth chamber; and
one or a plurality of cell culture plates positioned between the inlet and the outlet,
wherein the one or plurality of cell culture plates are arranged in a stacked configuration defining a plurality of flow channels through the cell growth chamber, and are oriented at an angle of from 0 to 75 degrees with respect to an axis of the cell growth chamber extending between the upper and lower end of the cell growth chamber.

Suitably, the axis is substantially perpendicular with respect to the upper and lower end.

Suitably, the one or plurality of cell culture plates are oriented parallel to the axis.

Suitably, a direction between the inlet and the outlet defines a flow direction, and wherein the flow direction substantially linear from the inlet to the outlet.

Suitably, the one or plurality of cell culture plates are oriented at an angle from 0 to 75 degrees with respect to the flow direction

Suitably, the plurality of cell culture plates are spaced apart by at least 1 millimetre.

Suitably, the plurality of cell culture plates are evenly spaced apart.

Suitably, the one or plurality of cell culture plates form a baffle for disrupting flow of cell culture medium from the inlet.

Suitably, the one or each of the plurality of cell culture plates extend from one side of the cell growth chamber to an opposing side of the cell growth chamber.

Suitably, the one or each of the plurality of cell culture plates comprises a coating for promoting cell adherence to thereto.

Suitably, the one or each of the plurality of cell culture plates comprises first and second cell culture surfaces.

Suitably, the first and second cell culture surfaces are substantially planar.

Suitably, the cell growth chamber further comprises a tapered outlet portion tapering inwardly from the one or plurality of cell culture plates towards the outlet.

Suitably, the tapered outlet portion is a conical base between the one or plurality of cell culture plates and the outlet.

Suitably, the tapered outlet portion tapers inwardly at an angle of from 25 to 65 degrees with respect to the axis of the cell growth chamber.

Suitably, the cell growth chamber further comprises a tapered inlet portion tapering outwardly from the inlet towards the one or plurality of cell culture plates.

Suitably, the one or plurality of cell culture plates are spaced apart from the inlet by a distance from 1 mm to 1 m.

Suitably, the one or plurality of cell culture plates are spaced apart from the outlet by a distance from 1 mm to 1 m.

Suitably, the cell growth chamber further comprises a cell trap configured to collect cells detached from the one or plurality of cell culture plates.

Described herein is also the use of the cell growth chamber for continuous bioprocessing of adherent cells.

Described herein is also a method for continuous bioprocessing of adherent cells using the cell growth chamber as described herein, the method comprising:
passing a cell culture medium through the inlet of the cell growth chamber;
collecting fluid from the outlet of the cell growth chamber, wherein the fluid comprises cell culture medium and cells detached from the one or plurality cell culture plates;
separating cells from the collected fluid.

It will be appreciated that the method for continuous bioprocessing of adherent cells may be carried out using a cell growth chamber including any of the features described above or any of the features of the cell growth chamber described herein.

For example, the cell growth chamber for continuous bioprocessing of adherent cells according to the first aspect may comprise:
an inlet positioned at an upper end of the cell growth chamber, wherein the inlet is couplable to a cell culture medium source;
an outlet positioned at a lower end of the cell growth chamber; and
one or a plurality of cell culture plates positioned between the inlet and the outlet,
wherein the one or plurality of cell culture plates are arranged in a stacked configuration defining a plurality of flow channels through the cell growth chamber, and are oriented at an angle of from 0 to 75 degrees with respect to an axis of the cell growth chamber extending between the inlet positioned at the upper end and the outlet positioned at the lower end of the cell growth chamber.

Suitably, the axis is substantially perpendicular with respect to the inlet positioned at the upper end and the outlet positioned at the lower end of the cell growth chamber.

The system for continuous bioprocessing of adherent cells of the present invention is associated with a number of advantages. For example, the system utilises a cell growth chamber that aids proliferation and harvesting of adherent cells with minimal human intervention. Furthermore, the system for continuous bioprocessing of cells is closed and sterile, and/or does not contain serum or other xenobiotics, thereby allowing the cells to be produced using good manufacturing practices. Furthermore, the system for continuous bioprocessing of cells may allow adherent cells to be harvested without the need to contact the adherent cells to cell anchoring substrate degradation agents (such as trypsin) which may have undesired effects on the cells, such as a reduction in cell activity.

### BRIEF DESCRIPTION OF THE DRAWINGS

Embodiments of the invention are now described, by way of example only, hereinafter with reference to the accompanying drawings, in which:

FIG. 1. shows a front view of a cell growth chamber;

FIG. 2. shows a side view of the cell growth chamber of FIG. 1;

FIG. 3a. shows an exploded view of another cell growth chamber;

FIG. 3b. shows sectional views of each component of the cell growth chamber of FIG. 3a;

FIG. 4. shows a method for continuous bioprocessing of adherent cells using the cell growth chamber of any of FIGS. 1 and 2, or 3a and 3b;

FIG. 5a. shows a system for continuous bioprocessing of adherent cells in a first mode of operation;

FIG. 5b. shows the system of FIG. 5a in a second mode of operation;

FIG. 6a. shows another system for continuous bioprocessing of adherent cells in a second mode of operation;

FIG. 6b. shows the system of FIG. 6a in a second mode of operation; and

FIG. 7. shows a method for continuous bioprocessing of adherent cells using the system of FIGS. 5a and 5b or 6a and 6b.

In the drawings, like reference numerals refer to like parts.

### DETAILED DESCRIPTION

Certain terminology is used in the following description for convenience only and is not limiting. For example, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

The present invention relates to a system for continuous bioprocessing of adherent cells. The term “adherent cells” as used herein refers to a homogenous or heterogeneous population of cells which are anchorage dependent, i.e. which require attachment to a surface in order to grow in vitro. Suitably, in the context of the present disclosure, the surface may be a cell culture plate, or it may be any other suitable surface, such as a microcarrier (e.g. a support matrix that allows for the growth of adherent cells in bioreactors). Adherent cells attach to the surface through an anchorage substrate such as integrin or other cell receptor. It will be appreciated that the surface (e.g. cell culture plate or microcarrier etc) may be made from or treated with a material that facilitates cell adhesion. Examples of materials that facilitate cell adhesion are described elsewhere in the present disclosure. Suitably, the adherent cells may be mammalian cells, for example human. Suitably, the adherent cells may be primary cells or immortalised cells. Merely by way of example, primary cells may be selected from the group consisting of myocytes, cardiomyocytes, epithelial cells, fibroblasts, keratinocytes, melanocytes, endothelial cells, osteoblasts, chondrocytes, adipocytes and mesenchymal stem cells. Merely by way of example, immortalised cells may be selected from the group consisting of HeLa cells, HEK 293 cells, 3T3 cells, A549 cells, VERO cells, CHO cells, OK cells, C2C12 and Ptk2 cells.

The systems described herein comprise a growth chamber. As would be clear to a person of skill in the art, a “growth chamber” refers to any suitable chamber or surface that is suitable for the growth of adherent cells. The “growth chamber” may therefore have any suitable type of surface and any suitable type of geometry. Appropriate growth chambers are well known in the art and include, but are not limited to, microcarriers, cell culture plates, cell culture containers, reaction vessels, as well as bioreactors. Suitable growth chambers may therefore include, but are not limited to hollow fibre, stirred tank, air-lift, bubble column or fluidised bed reaction vessels/bioreactors. Accordingly, although the invention is predominantly described below in the context of a growth chamber that comprises a cell culture plate, it is clear that any suitable growth chamber (e.g. a bioreactor) may also be used.

The systems described herein also comprise a cell trap. As would be clear to a person of skill in the art, a “cell trap” refers to any suitable means for trapping (e.g. capturing or restraining) cells. A “cell trap” may also be described as a cell separation device, or a means for preventing passage of cells. Suitable cell traps are readily identifiable by a person of skill in the art. For example, the cell trap may comprise a filter (e.g. using dead-end or crossflow/tangential fluid flow filtration), a centrifuge, acoustic separation or a microfluidic based system. Accordingly, although the invention is predominantly described below in the context of a cell trap that comprises a filter, it is clear that any suitable cell trap may also be used.

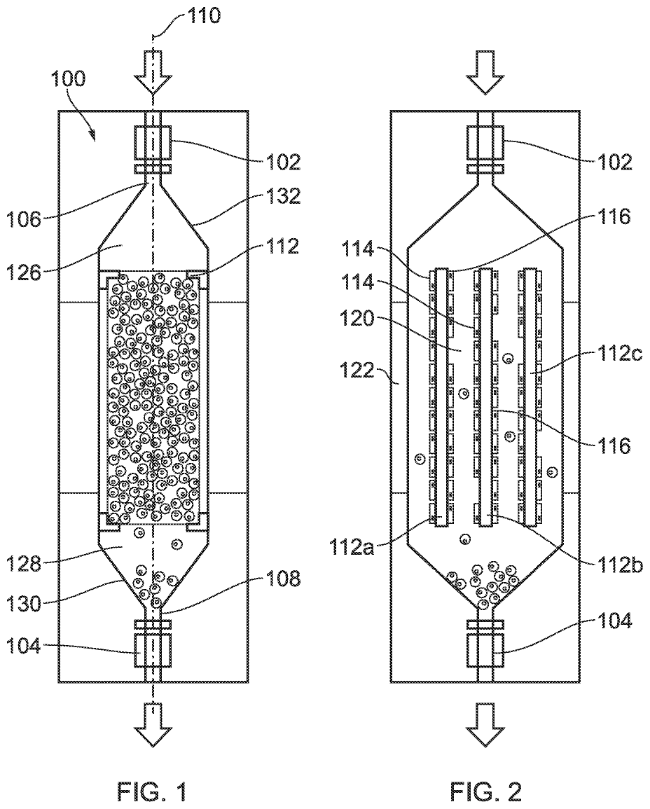

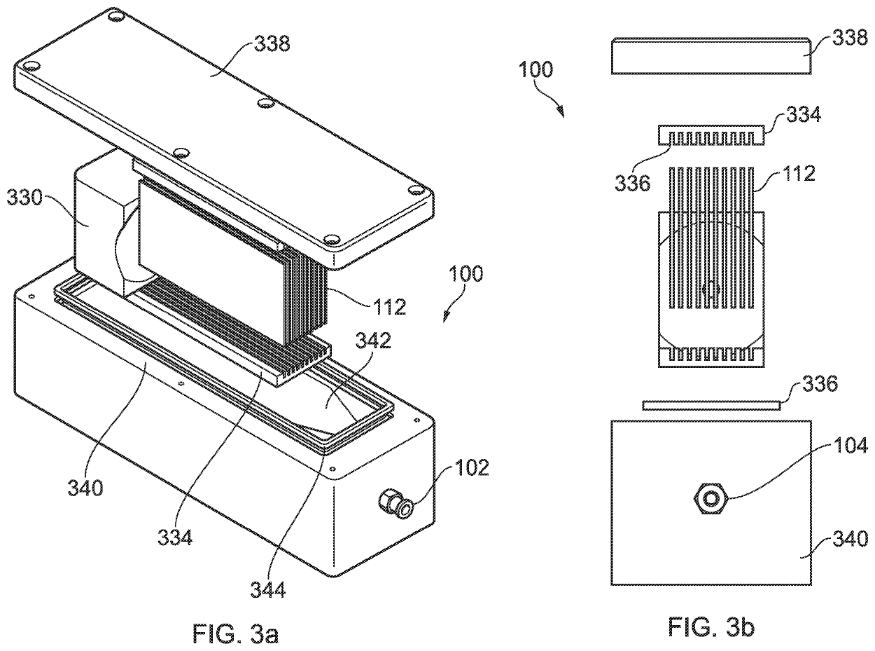

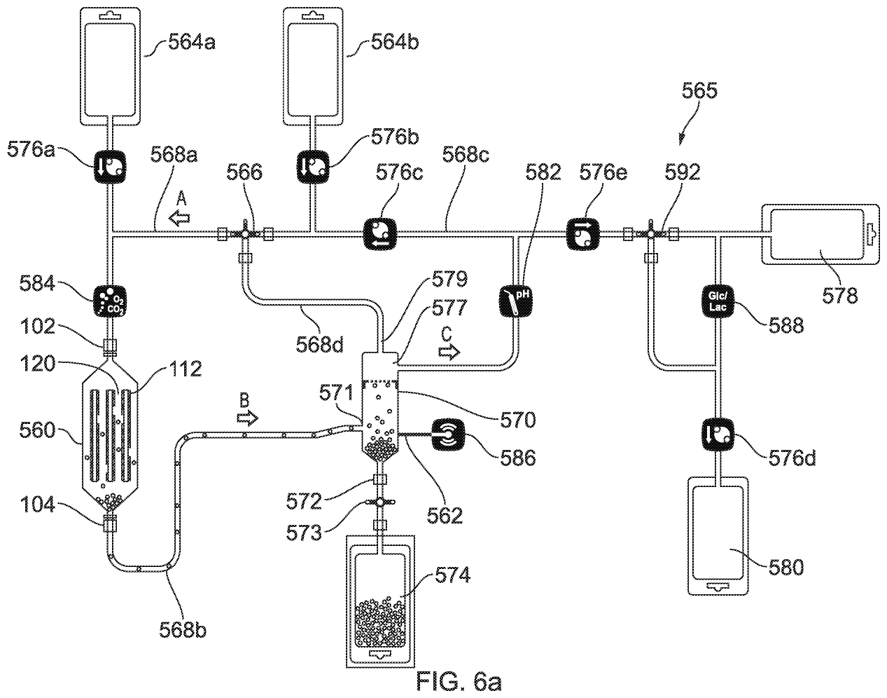

FIGS. 1 and 2 illustrate an example cell growth chamber 100. The cell growth chamber 100 has an inlet 102 and an outlet 104. The inlet 102 is adapted to be connected to a cell culture medium source, for example from an exterior reservoir (shown in FIG. 5). In this example the inlet 102 includes a luer-lock connector for connection to a fluid flow conduit, for example. The outlet 104 may be adapted to connect to a collection means, for example a conduit, or a cell trap. In this example the outlet 104 includes a luer-lock connector. The cell growth chamber 100 is thus easy to integrate into a fluid flow path of a bioprocessing system.

The term “cell culture medium” as used herein refers to a nutritive solution for cultivating live cells so as to allow the cell to proliferate. The cell culture medium may be a complete formulation, i.e., a cell culture medium that requires no supplementation to culture cells, or may be an incomplete formulation, i.e., a cell culture medium that requires supplementation or may be a medium that may supplement an incomplete formulation or in the case of a complete formulation, may improve culture or culture results. Various cell culture media will be known to those skilled in the art, who will also appreciate that the type of cells to be cultured may dictate the type of culture medium to be used.

Merely by way of example and not limitation, the cell culture medium may be selected from the group consisting of Dulbecco's Modified Eagle's Medium (DMEM), Ham's F-12 (F-12), Leibovitz's L-15 medium, RPMI-1640, Mesencult™ Basal Medium, Minimal Essential Medium (MEM), Basal Medium Eagle (BME), Ham's F-10, αMinimal Essential Medium (αMEM), Glasgow's Minimal Essential Medium (G-MEM), and Iscove's Modified Dulbecco's Medium (IMDM), or any combination thereof. Other media that are commercially available (e.g., from Thermo Fisher Scientific, Waltham, MA) or that are otherwise known in the art can be equivalently used in the context of this disclosure. Again, only by way of example, the media may be selected from the group consisting of 293 SFM, CD-CHO medium, VP SFM, BGJb medium, Brinster's BMOC-3 medium, cell culture freezing medium, CMRL media, EHAA medium, eRDF medium, Fischer's medium, Gamborg's B-5 medium, GLUTAMAX™ supplemented media, Grace's insect cell media, HEPES buffered media, Richter's modified MEM, IPL-41 insect cell medium, McCoy's 5A media, MCDB 131 medium, Media 199, Modified Eagle's Medium (MEM), Medium NCTC-109, Schneider's Drosophila medium, TC-100 insect medium, Waymouth's MB 752/1 media, William's Media E, protein free hybridoma medium II (PFHM II), AIM V media, Keratinocyte SFM, defined Keratinocyte SFM, STEMPRO® SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX bone marrow karyotyping medium, and KNOCKOUT D-MEM, or any combination thereof.

Suitably the cell culture medium may be serum-free. Suitably the cell culture medium may be glucose free.

The inlet 102 is positioned at an upper end 106 of the cell growth chamber 100 with respect to the outlet 104, which is positioned at a lower end 108 of the cell growth chamber 100. In this way, the inlet 102 is spaced apart from the outlet 104 at an opposed end of the cell growth chamber 100. In use, the cell culture medium may flow from the inlet 104 to the outlet 106 through the cell growth chamber 100. The inlet 104 and outlet 106 therefore define a flow direction therebetween. In some examples, the inlet 104 may be in line with the outlet 106 such that the flow of the cell culture medium is substantially linear.

The cell growth chamber 100 may include an axis 110 extending between the upper end 106 and lower end 108 of the cell growth chamber 100. The axis 110 may be substantially perpendicular with respect to the upper end 106 and the lower end 108. In this way, when the cell growth chamber 100 is in its standard orientation, the inlet 102 is positioned at the top of the cell growth chamber 100 and the outlet 104 is positioned at the bottom of the cell growth chamber 100. Therefore, the axis 100 may be substantially vertical in a standard orientation.

The cell growth chamber 100 has one or a plurality of cell culture plates 112 which are positioned between the inlet 104 and the outlet 106. The plurality of cell culture plates 112 may include from 2 to 100 plates, for example 10, 20, 30, 40, 50, 60, 70, 80, 90, 100 or more plates. It will be appreciated that the number of cell culture plates 112 may depend upon the size of the cell growth chamber 100. The plurality of cell culture plates 112 are arranged in a stacked configuration. The stacked plates 112 define a plurality of flow channels 120 between each of the plates 112. In other words, each pair of adjacent plates 112 have a space between them, which allows for the flow of cell culture medium through the cell growth chamber 100 in the flow direction. In this example the flow direction is substantially linear from the inlet to the outlet. In use, the cell culture medium enters the cell growth chamber 100 through the inlet 102 and then flows through the flow channels 120. In this way the plurality of cell culture plates 112 may form a baffle for disrupting flow of cell culture medium from the inlet 104.

The one or each of the cell culture plates 112 may be a substantially planar sheet. In this example the one or each of the cell culture plates 112 have a first cell culture surface 114 and a second cell culture surface 116. In a cell growth chamber 100 having a plurality of cell culture plates 112, the first cell culture surface 114 of a first cell culture plate 112a opposes a second cell culture surface 116 of a second, adjacent cell culture plate 112b, with a corresponding flow channel 120 therebetween.

The one or plurality of cell culture plates may extend laterally from one side of the cell growth chamber 100 to an opposing side of the cell growth chamber 100. In this way each flow channel 120 may extend between one side of the cell growth chamber 100 to the opposing side.

In this example, the cell culture plate(s) 112 are positioned along the longitudinal direction of the cell growth chamber 100. That is, the cell culture surfaces 114, 116 of the cell culture plate(s) extend longitudinally from an upper portion 126 of the cell growth chamber 100 to a lower portion 128 of the cell growth chamber 100. The cell culture plate(s) 112 may be spaced apart from one or both of the inlet and the outlet 104. This provides space for the fluid to disperse over the cell culture plate(s) 112 more evenly. For example, the cell culture plate(s) 112 may be positioned between 1 millimetre and 1 meter from the inlet 102. Suitably, the cell culture plate(s) 112 may be positioned between from about 10 millimetres to about 100 millimetres, for example about 50 millimetres from the inlet 102. Further, the cell culture plate(s) 112 may be positioned between 1 millimetre and 1 meter from the outlet 104. Suitably, the cell culture plate(s) 112 may be positioned between from about 10 millimetres to about 100 millimetres, for example about 44 millimetres from the outlet 104.

The normal (vertical) orientation of the cell growth chamber 100 allows optimal cell collection by utilising gravity to support the flow direction of the cell culture medium towards to the outlet.

The cell culture plate(s) 112 are positioned such that cells may adhere to both the first and second cell culture surfaces 114, 116 of the plate(s). Suitably, the cell culture plate(s) are oriented at an angle from 0 to 75 degrees from the axis 110 extending between the upper end 106 and lower end 108 of the cell growth chamber 100. Aptly, the cell culture plate(s) 112 are oriented at an angle from 0 to 45 degrees from the axis 110. In this example, the cell culture plate(s) 112 are substantially aligned with the axis 110. That is, the cell culture plate(s) 112 are oriented at an angle of 0 degrees with respect to the axis 110. In this way, the one or plurality of cell culture plates 112 may be parallel to the flow direction of the cell culture medium.

Suitably, the cell culture plate(s) 112 may be oriented at an angle from more than 0 to 75 degrees, for example more than 0 to 45 degrees from the axis 110 extending from the upper end 106 and lower end 108 of the cell growth chamber. Merely by way of example, the cell culture plate(s) may be oriented at an angle from 5 degrees to 75 degrees or 5 to 45 degrees from the axis 110 extending between the upper end 106 and lower end 108 of the cell growth chamber 100.

The one or plurality of cell culture plates 112 may be arranged within the cell growth chamber 100 such that the cell culture surface of the plate, or a first plate 112a in the stack, is parallel to a wall 122 of the cell growth chamber 100. The wall 122 of the cell growth chamber 100 and the plate, or the first cell culture plate 112 in the stack, may therefore also define a flow channel 120 therebetween. Similarly, the cell culture surface of the plate, or a last plate 112c in the stack, is parallel to an opposite section of the wall 122 of the cell growth chamber 100. The wall 122 of the cell growth chamber 100 and the plate, or the last cell culture plate 112c in the stack, may therefore also define a flow channel 120 therebetween.

The cell culture plates 112a-c may be spaced apart substantially equidistantly from one another. That is, the distance between the first cell culture surface 114 of one cell culture plate 112a an opposed second cell culture surface 116 of an adjacent cell culture plate 112b may be consistent between plates such that the flow channel 120 therebetween has a constant cross-sectional area along its length. In this example, the cell culture plates may be spaced apart by around 0.1 millimetres to 10 millimetres. Aptly, the cell culture plates 112 are spaced apart by around 1 millimetre to 5 millimetres. The cell culture plates 112 may each be spaced apart equally such that flow velocity through each fluid flow path in the cell growth chamber 110 is substantially equal. Alternatively, the cell culture plates 112a-c may be not spaced apart equidistantly. Suitably, when the cell culture plates 112a-c are not spaced equidistantly, the space between the cell culture plates 112a-c may increase (for example gradually increase) from the central or substantially central cell culture plate 112. In this context, the central cell culture plate may be the plate that is most closely position to the axis 110.

It will be appreciated by a person skilled in the art, that the flow velocity through each fluid flow path in the cell growth chamber 100 may be such that it does not result in excessive shear stress. The term “excessive shear stress” as used herein refers to shear stress that results in inhibition or reduction of cell proliferation, cell damage, cell death and/or increased cell detachment rate as compared to cells grown in static cell culture. In this context, “static cell culture” refers to cell culture where there is no flow velocity in the cell growth chamber, as opposed to “flow cell culture” where there is flow velocity through a fluid flow path in the cell growth chamber described herein.

Suitably, excessive shear stress may be more than about 0.1 Pa, more than about 0.2 Pa, more than about 0.3 Pa, more than about 0.4 Pa, or more than about 0.5 Pa. More suitably excessive shear stress may be more than about 0.1 Pa. Accordingly, the cell culture growth chamber 110 may be configured such that in use, the shear stress is 0.1 Pa or less, 0.2 Pa or less, 0.3 Pa or less 0.4 Pa or less or 0.5 Pa or less. It will also be appreciated by the person skilled in the art that excessive shear stress may depend upon cell type.

The cell culture plate(s) 112 are configured to provide an environment to which cells can adhere to and proliferate. The cell culture plate(s) 112 may be made from glass or plastic. Suitably, the cell culture plate(s) 112 may comprise a material that facilitates cell adhesion. Herein such a material that facilitates cell adhesion may be also referred to as “adherent material”.

Merely by way of example, the material that facilitates cell adhesion may be selected from the group consisting of a polyester, a polypropylene, a polyalkylene, a polyfluorochloroethylene, a polyvinyl chloride, a polyvinyl fluoride resin, a polystyrene, a polysulfone, a polyurethane, a polyethyene terephtalate, a cellulose, a glass fiber, a ceramic particle, a matrigel, an extracellular matrix component, a collagen, a poly L lactic acid, a dextran, an inert metal fiber, silica, natron glass, borosilicate glass, chitosan, or a vegetable sponge. Suitably, the cellulose may be cellulose acetate. The extracellular matrix component may be one or more of fibronectin, vitronectin, chondronectin, or laminin. Suitably, the adherent material is electrostatically charged. Suitably, the adherent material may be coated with collagen or gelatin. Suitably, the adherent material may comprise a peptide amphiphile (PA), such as that described in Miotto et al, Developing a Continuous Bioprocessing Approach to Stromal Manufacture, ACS Applied Materials & Interfaces 2017 9 (47), 41131-41142. In this example, the adherent material may be applied to the first cell culture surface 114 and the second cell culture surface 116 of the or each cell culture plate. It will be appreciated that within the same cell growth chamber 100, some or all of the cell culture plates 112 and/or cell culture cell culture surfaces 114, 116 may comprise the same or different adherent material.

The cell culture plate(s) 112 may be seeded with adherent cells prior to being placed in the cell growth chamber 100. Suitably, the adherent cells may be seeded on one or both surfaces of the cell culture plate(s).

Once the cell culture plate(s) 112 are placed within the cell growth chamber 100, the first cell culture surface 114 and second cell culture surface 116 are both exposed to the cell culture medium enabling adherent cells to grow on one or both surfaces of the cell culture plate(s) 112. Once the cells have detached from the cell culture plate(s) 112 they flow along the flow channel in the cell culture medium towards the outlet 104.

In some examples the cell growth chamber 100 may include a cell trap (shown separately in FIG. 5a for example). The cell trap is configured to capture cells detached from the cell culture plate(s) with a filter element configured to allow passage of cell culture medium and block passage of detached cells. The cell trap will be described in detail with reference to FIG. 5a.

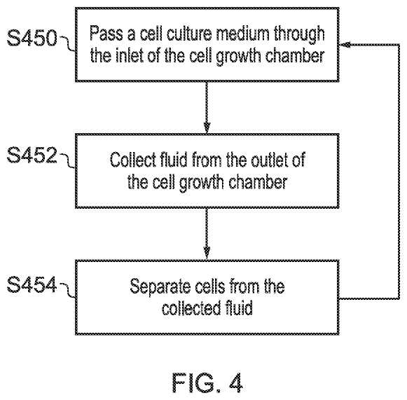

The cell growth chamber may include a tapered outlet portion 130. The tapered outlet portion 130 tapers inwardly from the one or plurality of cell culture plates 112a-c towards the outlet 104. In this way, the tapered outlet portion 130 directs detached cells and cell culture medium towards the outlet. This can help to avoid a build-up of detached cells on a bottom wall of the cell growth chamber 100, thereby improving cell collection. In this example, the tapered outlet portion 130 is integral with the cell growth chamber walls. The tapered outlet portion 130 may taper inwardly at an angle of from 25 to 65 degrees with respect to the axis 110. In some examples a tapered outlet portion 130 may be a separate part, as shown in FIG. 3a. For example, the tapered outlet portion 130 may include a conical base portion 330.

In some examples the cell growth chamber 100 may also include a tapered inlet portion 132 tapering outwardly from the inlet 102 towards the one or plurality of cell culture plates. The tapered inlet portion 132 may help to more evenly distribute the cell culture medium entering the cell growth chamber 100 from the inlet 102.

FIGS. 3a and 3b illustrate a further example of the cell growth chamber 100 assembly. For example, the cell culture plate(s) 112 may be held in place with a holding element 334 on each side of the cell chamber 100. The holding element 334 may hold the plurality of cell culture plates 112 spaced apart. The holding element 334 may include one or a plurality of slots 336 for accepting the one or plurality of cell culture plates. The width of the slots 336 may correspond to the width of the cell culture plate(s) 112. As such, each slot in the holding element 334 may retain a corresponding cell culture plate 112 in position. The holding element 334 may be any suitable material, for example silicone.

As shown in FIG. 3a the cell growth chamber 100 may be configured to allow access to the cell culture plate(s) 112. In this way, the inside of the chamber may be cleaned or the cell culture plate(s) may be replaced as desired.

In this example the cell growth chamber includes a main body portion 340 and a removable side wall portion 338. The removable side wall portion 338 is configured to couple with the main body portion 340 to define an internal chamber volume therebetween. For example, the main body portion 340 portion may include a cavity 342 and the side wall portion 338 may form a lid to close the cavity 342.

The side wall portion 338 may removably couple with the main body portion 340 via any suitable means, for example screws. In this example a sealing element 344 is positioned around the periphery of the cavity 342, to fluidly seal the internal chamber volume when the side wall portion 338 is coupled with the main body portion 340. The sealing element 344 may be a silicon O-ring for example. In some examples, the side wall portion 338 and the main body portion 340 may be insulated to help regulate the temperature of the cell growth chamber 100.

The conical base portion 330 may be positioned inside the cavity 342 adjacent the outlet of the chamber. The conical base portion 330 may be held in position by a friction fit between the main body portion 340 and the removable side wall portion 338. An upper surface of the conical base portion 330 may also include first and second substantially planar abutment surfaces configured to abut against the holding elements 334 on either side of the chamber. In this way, the conical base portion 330 may be retained in position adjacent to the outlet 104 of the chamber.

The cell culture plate(s) 112 are positioned in the cavity 342 and are held in place in a spaced apart configuration with the holding element 334 as discussed above. In use, the cell culture medium can be introduced through the inlet 102 such that it flows through the cavity 342 passing over the surfaces of the cell culture plate(s) 112 and is then directed by the conical base portion 330 towards the outlet 104.

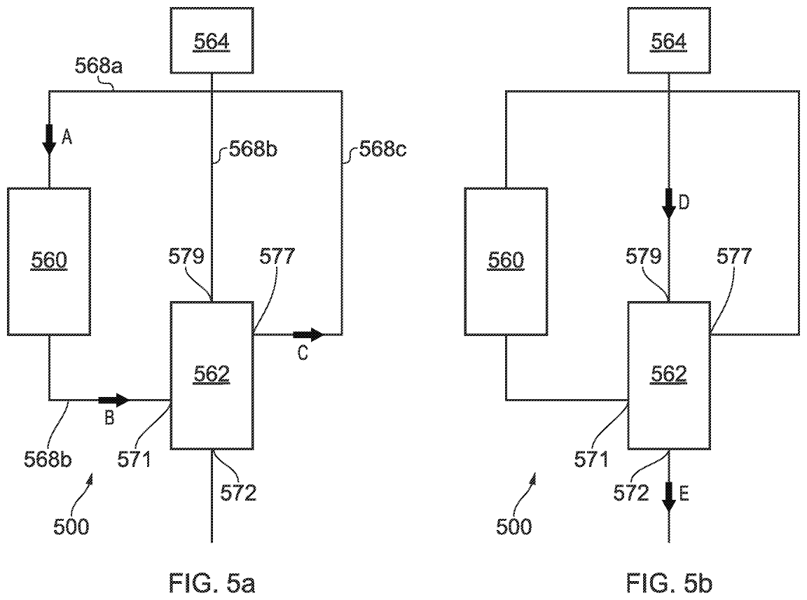

FIG. 4 shows an example of a method for continuous bioprocessing of adherent cells. As a first step S450 cell culture medium is passed through the inlet 102 of the cell growth chamber 100. The cell culture medium will then pass through the flow channels 120 towards the outlet, with some cells detaching from the cell culture plate(s) 112 and flowing within the cell culture medium towards the outlet. This fluid may then be collected from the outlet 104 in step S452.

In step S454 the cells are separated from the fluid. That is, detached cells may be collected by a cell trap or other suitable collection mechanism. In this example, the fluid output from the outlet 104 is passed through the cell trap during the medium recycling, the cell trap preventing detached cells re-entering the cell growth chamber. The detached cells may therefore be collected in the cell trap and separated from the fluid. The fluid may then be returned to the inlet 102 and passed through the cell growth chamber 100 again. That is, the cell culture medium may be recycled. Additional medium and/or nutrients may be added to the cell culture medium during the recycling.

FIGS. 5a and 5b illustrate a system 500 for continuous bioprocessing of adherent cells. The system 500 includes a cell growth chamber 560 and a cell trap 562, which is in fluid communication with the cell growth chamber 560. The cell growth chamber 560 may be configured similarly to the cell growth chamber 100 as described above, or may be another suitable chamber for the growth of cells.

The system 500 further includes a fluid reservoir 564. The fluid reservoir 564 may be a cell medium reservoir and is fluidly connected to the cell growth chamber 560 and the cell trap 562. In this way fluid, for example a cell culture medium, can be directed through the system 500. In this example, the fluid reservoir 564, cell growth chamber 560 and cell trap 562 are fluidly connected via conduits 568a-d. In other examples, one or more of the fluid reservoir 564, the cell growth chamber 560, and the cell trap 562 may be directly fluidly connected, without the need for conduits 568a-d extending between.

The system 500 is configured to operate in a first mode of operation as shown in FIG. 5a and a second mode of operation as shown in FIG. 5b. The first mode of operation allows for cell growth within the chamber 560 and the second mode of operation allows for harvesting of cells collected in the cell trap 562. The system 500 is configured to selectively switch between the first mode of operation and the second mode of operation.

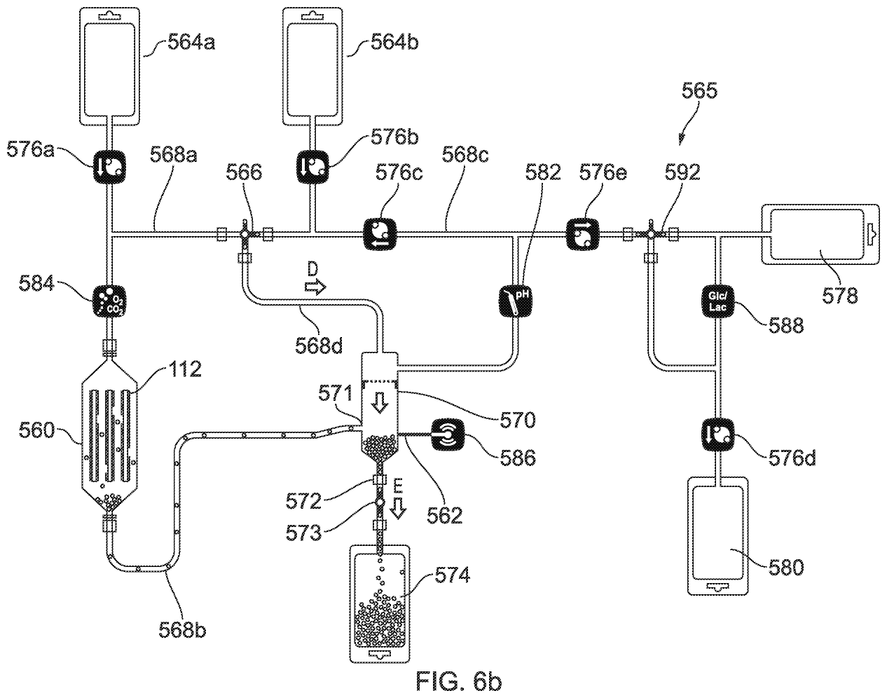

In the first mode of operation illustrated in FIG. 5a, the system is configured to direct fluid through the cell growth chamber 560. In use, the fluid flows from the fluid reservoir 564 to the cell growth chamber 560 as illustrated by arrow A. The fluid then passes through the growth chamber 560 and flows to the cell trap 562 as illustrated by arrow B. The fluid may then exit the cell trap 562 ready for recirculation through the system 500, as illustrated by arrow C. Alternatively, the system 500 may be configured to direct at least a portion of the fluid from the cell trap 562 towards a fluid waste circuit 565 as described later with reference to FIGS. 6a and 6b.

The system 500 may also be configured to allow additional fluid from the reservoir 564 to enter the system as needed to maintain a desired volume of circulating fluid in the system 500.

In the second mode of operation, illustrated in FIG. 5b, the system 500 is configured such that fluid from the fluid reservoir 564 by-passes the cell growth chamber 560. That is, the system is configured to direct fluid from the fluid reservoir 564 directly to the cell trap 562 without passing through the cell growth chamber 560 as indicated by arrow D. The cells collected in the cell trap 562 can be then flushed from the cell trap 562 with the cell culture medium through a cell outlet 572 as indicated by arrow E. In this way, cells can be harvested periodically from the cell trap 562 and the system 500.

Referring now to FIGS. 6a and 6b, the system 500 is shown in more detail. FIG. 6a illustrates the system 500 in the first mode of operation and FIG. 6b illustrates the system 500 in the second mode of operation.

In the first mode of operation, the system 500 is configured such that fluid is pumped from the fluid reservoir 564 towards the cell culture chamber 560. In this example the fluid reservoir 564 includes a separate glucose reservoir 564a and a cell culture medium reservoir 564b.

The system includes at least one pump configured to direct fluid around the system. In this example, the system includes a plurality of pumps 576a-e. Each of the pumps may be any suitable pump, for example a perfusion pump.

In this example the system 500 includes a glucose pump 576a for directing glucose from the glucose reservoir 564a towards the cell culture chamber 560; a cell medium pump 576b for directing cell medium from the cell culture medium reservoir 564b towards either the cell culture chamber 560 or the cell trap 570; a recirculation pump 576c between the cell trap 562 and the reservoir 564; a PBS pump 576d (described below); and a waste pump 576e for directing the fluid into the waste circuit 565. It will be appreciated that any number of pumps can be incorporated into the system for directing the fluid flow. Aptly, there is no fluid pump between the cell growth chamber 560 and the cell trap 562, such that detached cells do not flow through a pump, which can cause damage to the cells. In some examples, the pumps may pulse the fluid flow into the cell growth chamber 560. The pulse rate may vary for example, from being continuous to once a day. Suitably, the pulse rate of the glucose pump may be such that the level of glucose in maintained in the cell culture medium. It will be appreciated that the pulse rate may depend on the size and number of the cell culture plate(s) 112, cell count, cell type, and/or amount of glucose consumed by the glucose sensor 588.

In this example, system 500 includes a gas exchanger between the fluid reservoir 564 and the cell culture chamber 560. In use, fluid from the fluid reservoir passes through the gas exchanger 584 before reaching the cell culture chamber 560. The gas exchanger 584 may ensure that the cell culture medium entering the cell growth chamber 560 is suitably aerated and/or has a suitable pH to support cell growth within the cell culture chamber 560. Suitably, the gas exchanger 584 may increase oxygen levels within the cell culture medium, and/or decrease carbon dioxide levels within the cell culture medium entering the cell culture growth chamber 560.

As the fluid passes through the cell growth chamber 560, it aids in removal of the cells that are loosely adhered or no longer adhered to the cell culture plate(s) 112. The removed cells are carried in the fluid towards the growth chamber outlet 104. As such, fluid exiting the cell growth chamber 560 may therefore include cells that have detached from the cell culture plate(s).

In this example, the cell growth chamber includes an inlet 102, an outlet 104, and one or a plurality of cell culture plates 112 positioned between the inlet 102 and the outlet 104. The plurality of cell culture plates 112 are arranged in a stacked configuration defining a plurality of flow channels 120 through the cell growth chamber. The cell culture plate(s) 112 are oriented such that cells detached from the cell culture plates travel along the flow channels 120 and through the outlet 104. In use, the system 500 may be orientated such that the outlet 104 is positioned vertically below the inlet 102 so as to take advantage of gravitational pull to aid in the directional flow of the fluid. Additionally, the gravitational pull may aid detachment of the cells from the cell culture plate(s) 112, in particular those cells that are loosely attached to the cell culture plate(s) 112 and/or detached from cell culture plate(s) 112 but attached to other adherent cells attached to the cell culture plate(s) 112.

Continuous cycling of the fluid through the growth chamber 560 allows for continuous cell growth. That is, as the cells attached to the culture plate(s) (or attached to a microcarrier, in a bioreactor for example) proliferate and reach confluency, a proportion of the cells may become more loosely attached or detached from the cell culture plate(s) 112 (or attached to a microcarrier, in a bioreactor for example), thereby providing space for the attached cells to continue proliferating.

The cells may become more loosely attached or detached from the cell cultures plate(s) 112, for example, as a result of decreased expression and/or degradation of cell anchorage substrates. Generally speaking, cells with a higher passage number may have decreased expression and/or increased degradation of cell anchorage substrates as compared to cells with lower passage numbers. Such reduced expression and/or increased degradation may result in cells having a higher passage number to be outcompeted for space on the cell culture plate(s) 112 by cells having lower passage numbers. Cells may also become more loosely attached or detached from the cell culture plate(s) 112 as a result of the cells being contacted with a cell anchorage substrate degradation agent. By way of example, the cell anchorage substrate degradation agent may be trypsin, papain, elastase, hyaluronidase, collagenase type 1, collagenase type 2, collagenase type 3, collagenase type 4, dispase, all-trans retinoic acid, or a combination thereof. The presence of all-trans retinoic acid in the medium may result in degradation of the cell anchoring protein MMP1. Degradation of the cell anchorage substrate may occur steadily over-time allowing for cells that have been exposed to the degradation agent for longer periods of time to become loosely detached or detached as compared to cells than have been not been exposed or have been exposed for shorter periods of time. At the same time, cells that have not been exposed, or have been exposed to the degradation agent for shorter periods of time may remain attached and proliferate. The system may include a cell transport conduit 568b for fluidly connecting the outlet of the cell growth chamber 560 to a cell inlet 571 of the cell trap 562. In use, the detached cells may flow within the fluid from the cell growth chamber 560 to the cell trap 562 via the cell transport conduit 568b. In this example, fluid flow along the conduit 568b between the cell growth chamber 560 and the cell trap 562 is facilitated by back pressure and optionally gravity. In this way, damage to cells that can be caused by faster fluid flows can be avoided.

The cell trap 562 is configured to allow fluid to pass through, whilst trapping the detached cells. For example, the cell trap 562 may include a filter 570 configured to prevent passage of detached cells. The filter 570 may be a mesh, or other suitable filter material, having apertures sized to allow passage of fluid and prevent passage of detached cells. This enables collection of the detached cells and prevents them from being recirculated through the system, where they could be damaged when passing through the fluid pumps, for example. In addition, the cell trap 562 may be configured to allow cells collected in the cell trap to settle in a lower portion of the cell trap. The cell trap 574 further includes a fluid outlet 577 positioned on an opposite side of the filter to the cell inlet 571.

The system is configured such that fluid passing through the filter 570 may flow towards a waste circuit 565 or be recirculated through the cell growth chamber 560. A pH sensor may be located in the fluid passage adjacent the fluid outlet 577 of the cell trap. The fluid may pass through a pH sensor and the pH sensor may determine if the pH of the fluid is of a sufficient level to allow the fluid to be recirculated through the cell growth chamber 560. For example, acceptable levels of pH may be from 7 to 8, suitably 7.2 to 7.6, more suitably about 7.4, to ensure the fluid does not prevent growth of cells in the cell growth chamber. If the pH is within acceptable levels the fluid is recirculated through the cell growth chamber 560. The system may include a recirculation pump 576c positioned in a fluid pathway between the fluid outlet of the cell trap 570 and the cell growth chamber 560. The recirculation pump 576c may be selectively activated when recirculation of the fluid is desired (e.g. when the pH is at a suitable level), to help direct the fluid back through the cell growth chamber 560.

If the pH is determined to be outside the acceptable range, the system 500 is configured to direct fluid from the cell trap 570 towards the waste circuit 565. For example, the system 500 may be configured to selectively activate a waste pump 576e in response to a pH reading outside the acceptable range. The waste pump 576e is configured to direct at least a portion of the fluid from the fluid outlet 577 of the cell trap 570 to the waste circuit 565. The waste circuit 565 may include an optional glucose sensor 588. The system 500 is configured to selectively activate a valve 592 to direct the fluid directly into the waste collection chamber 578 or pass the fluid through the glucose sensor 588.

The glucose sensor may be enzyme or non-enzyme based. An enzyme based glucose sensor may employ, for example glucose oxidase enzyme (GOx) that oxidizes glucose and generates detectable compounds such as O₂, CO₂, or H₂O. Other examples of enzyme based glucose sensors will be known to those skilled in the art. When the fluid has been passed through the glucose sensor 588 the system 500 may activate a flush protocol. That is the system 500 may activate the PBS pump 576d to direct phosphate buffered saline buffer (PBS) through the sensor.

The flush protocol may extend the life of the glucose sensor 588. In particular, the flush protocol may extend the life of an enzyme based glucose sensor because it prevents from the cell culture media remaining in contact with the glucose sensor and reacting with the enzymes. The data from the glucose sensor 588 may allow the system 500 to alter the glucose levels in the fluid by switching on/off the glucose pump 576a associated with the glucose reservoir to add glucose to the fluid in the system.

Referring now to FIG. 6b, in order to harvest the cells collected in the cell trap 562 the system 500 is configured to switch to the second mode of operation. In the second mode of operation, the system is configured to direct cell culture medium from the cell culture medium reservoir 564b to the cell trap 570 and through the cell outlet 572 to thereby harvest cells collected in the cell trap 570.

To switch between the first and second modes of operation the system 500 may include a valve assembly with a first valve 566. The valve assembly is configured to selectively switch between a first configuration corresponding to the first mode of operation and a second configuration corresponding to the second mode of operation.

In this example, the first valve 566 is configured such that in a first configuration the first valve 566 prevents flow of fluid from the cell culture medium reservoir 564b directly to the cell trap 570 without passing through the cell growth chamber 560. In the first configuration, the first valve 566 allows flow of fluid from either the cell trap 570 or the cell culture medium reservoir 564b towards the cell culture chamber 560.

In this example, the first valve 566 is positioned at a junction between three conduits. The first conduit 568a extends between the valve 566 and the cell growth chamber 560 and may be referred to as a growth chamber inlet conduit. The second conduit 568d extends between the valve 566 and a fluid inlet 579 of the cell trap 570 and may be referred to as a by-pass conduit. The third conduit 568c extends between the fluid outlet 577 of the cell trap 570 and the valve 566 and may be referred to as a recirculation conduit.

To switch between the first and second configuration, the valve may be configured to allow fluid flow between two of the three conduits 568a,b,d, while preventing fluid flow to the other of the three conduits 568a,b,d. For example, in the first configuration shown in FIG. 6a, the valve 566 is configured to enable fluid flow from the recirculation conduit 568c to the growth chamber inlet conduit 568a, whilst preventing fluid flow to the by-pass conduit 568d.

In the second configuration shown in FIG. 6b, the valve 566 is configured to prevent fluid flow along the growth chamber inlet conduit 568a and enable fluid flow along the by-pass conduit 568d. In this way, the valve 566 is able to direct flow from the reservoir 564 directly to the cell trap 562 by closing off the growth chamber inlet conduit 568a extending between the reservoir 564 and the cell growth chamber 560 while opening a by-pass conduit 568d to directly link to the cell trap 562 to the reservoir 564. In this way, the fluid can flow directly to the cell trap 562. This flow of fluid can then help to direct detached cells into the collection container 574.

The valve assembly may further include a second valve 573 between the cell trap 562 and the collection container 574. In the first mode of operation the second valve prevents fluid communication between the collection container 574 and the cell trap 562. In the second mode of operation the second valve 573 is opened such that the cell trap 562 is in fluid communication with the collection container 574. In the second configuration, the fluid from the cell culture medium reservoir 564 therefore encourages cells to flow from the cell trap into the collection container 574 via the cell outlet 572. In use, for some examples the collection container 574 may be positioned below the cell trap 562 such that gravity helps to direct the cells into the collection container 574 in addition to the fluid flow from the reservoir 564.

The collection chamber 574 is aptly detachable from the system 500. For example, a luer-lock connector may be provided adjacent to the second valve 573 for connection with the collection chamber 574. When the second valve 573 is in the closed configuration (as shown in FIG. 6a), the collection container 574 may be disconnected and replaced as desired, for example when it has reached capacity.

In some examples the cell trap 562 may further include an actuator 586. The actuator may be configured to impart mechanical vibrations to the cell trap 562 to thereby encourage cells to move towards the cell outlet 572. The actuator may be a piezoelectric transducer. The actuator may be configured to operate in the second mode of operation, and may be configured to selectively actuate.

The cell trap 562 may include a sensor (not shown), which is able to monitor the quantity of cells collected in the cell trap 562. When the quantity of cells reaches a predetermined threshold the system 500 may switch from the first mode of operation to the second mode of operation.

The system 500 may include a controller (not shown). The controller may be configured to collect data from at least one of the sensors distributed around the system 500 and react by changing the operational mode, and/or activating one or more of the valves, pumps, or actuators. The controller may therefore include a receiver for receiving data from the system components, and a decision module. Similarly each of the sensors, valves, pumps, and actuators may include transmitters and receivers to send and receive data. In some examples the system may be controlled wirelessly, wholly or in part. The controller may therefore act to maintain properties of the circulating fluid, for example pH and glucose concentration, within predetermined acceptable ranges. Further, the system 500 may be automated such that selective switching between operational modes is automatic. In this way, the system 500 may be capable of operating with minimal human input.

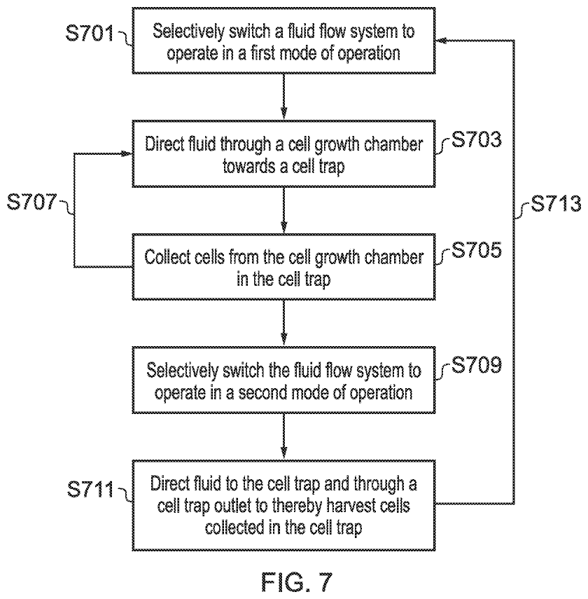

Referring now to FIG. 7, a method of operation for the system 500 is shown. In a first step S701 the system 500 is selectively switched to operate in the first mode of operation. Fluid in the system is directed through the cell growth chamber 560 in a direction towards a cell trap, in step S703. As the fluid is directed through the cell growth chamber 560 it aids the removal of cells that are loosely attached or detached from the cell culture plate(s) 112 from the cell growth chamber 560 to the cell trap 562. The cells are collected in the cell trap 562 in step S705. This process may be repeated S707. During the first mode of operation the properties of the fluid may be monitored (e.g. pH and glucose concentration), and the system 500 may selectively activate the inputs (e.g. glucose and cell culture medium) and switch between recirculating fluid from the cell trap and passing fluid to the waste circuit.

The system 500 is then selectively switched to operate in the second mode of operation in step S709. The selective switch S709 may be triggered when the volume of cells collected within the cell trap 562 reaches a predetermined threshold value, for example. The fluid is then directed through the cell trap 562 such that cells contained within the cell trap 562 can be harvested S711. For example, the cells are passed through a cell outlet 572 and into the collection chamber 574. In some examples, the cells may be transferred into the collection chamber 574 by gravity. The entire process may then be repeated S713.

Various modifications to the detailed arrangements as described above are possible without departing from the scope of the claims.

Although described above as having single inlets and outlets, the cell growth chamber may include multiple inlets and or outlets at the top and bottom portions respectively. In this way the cell growth chamber may include a plurality of “mini-chambers” stacked adjacent within one system.

Although the cell trap is described above as a separate component to the cell growth chamber, it will be appreciated that the cell trap may be integral with or directly fluidly connected to the cell growth chamber, without the need for a conduit extending between.

Additional or alternative reservoirs to the glucose and cell culture medium reservoirs may be included in the system depending on the desired conditions for cell growth.

The harvested cells may be plated out into individual wells by use of a robotic liquid handler. In some examples harvested cells may be exposed to different growth conditions. Merely by way of example, if the harvested cells are stem cells, they may be exposed to culturing conditions that may lead to stem cell differentiation.

Although the cell culture plate(s) are described above as having substantially planar first and second cell culture surfaces, it will be appreciated that planar surfaces are not essential and that the cell culture surfaces may be curved, for example, or include undulations.

In the context of the present invention the purpose of the glucose reservoirs may be to add glucose to the fluid in the system and thereby provide an energy supplement to the cells. However, it will be readily appreciated by a person skilled in the art, that it may be desirable to culture cells in an energy supplement other than glucose, for example galactose. Accordingly, the system for continuous bioprocessing of cells as described herein may comprise an energy supplement reservoir 564a and a respective energy supplement pump 576a and energy supplement sensor 588.

The above described system having a first and second operational mode allows for continuous bioprocessing of adherent cells in which the cells can continually proliferate in the cell culture chamber and be periodically harvested from the system. As touched upon elsewhere in the present disclosure, the present invention may have a number advantageous such as it involves minimal human intervention. In the context of the present invention, such minimal human intervention is also relevant to the step of cell harvesting, which normally requires operator input in order to detach the cells. For example, the growth chamber enables the adherent cells to proliferate and progressively detach upon reaching confluency due to the forces of gravity and cell culture medium flow. Beneficially, detachment of the cells does not require cell anchoring substrate degradation agents (such as trypsin). Furthermore, the growth chamber may be used in a system for continuous bioprocessing of cells that is closed and sterile, and/or does not contain serum or other xenobiotics, thereby allowing the cells to be produced using good manufacturing practices.

It will be clear to a person skilled in the art that features described in relation to any of the embodiments described above can be applicable interchangeably between the different embodiments. The embodiments described above are examples to illustrate various features of the invention.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A system for continuous bioprocessing of adherent cells, the system comprising:
a cell growth chamber;
a cell trap in fluid communication with the cell growth chamber, the cell trap including a cell outlet; and
a fluid flow system configured to selectively switch between a first mode of operation and a second mode of operation;
wherein, in the first mode of operation the fluid flow system is configured to direct fluid through the cell growth chamber towards the cell trap such that cells from the cell growth chamber are collected in the cell trap, and
in the second mode of operation the fluid flow system is configured to direct fluid to the cell trap and through the cell outlet to thereby harvest cells collected in the cell trap.

2. The system of claim 1, wherein the system further comprises at least one reservoir for introducing fluids into the fluid flow system.

3. The system of claim 1, wherein in the first mode of operation the fluid flow system is configured to direct fluid from the cell trap towards a fluid waste outlet or back through the cell growth chamber.

4. The system of claim 1, wherein the cell trap comprises a filter configured to prevent passage of cells towards the fluid waste outlet or back through the cell growth chamber.

5. The system of claim 1, wherein in the second mode of operation the fluid flow system is configured such that fluid by-passes the cell growth chamber.

6. The system of claim 1, further comprising a valve assembly configured to selectively switch between a first configuration corresponding to the first mode of operation and a second configuration corresponding to the second mode of operation.

7. The system of claim 6, wherein in the first configuration the valve assembly is configured to prevent flow of fluid directly to the cell trap without first passing through the cell growth chamber.

8. The system of claim 6, wherein in the first configuration the valve assembly is configured to allow flow of fluid from a fluid reservoir to the cell growth chamber.

9. The system of claim 6, wherein in the second configuration the valve assembly is configured to prevent flow of fluid from a fluid reservoir to the cell growth chamber.

10. The system of claim 9, wherein in the second configuration the valve assembly is configured to allow flow of fluid from a fluid reservoir directly to the cell trap.

11. The system of claim 6, wherein the valve assembly comprises a first valve configured to selectively control flow of fluid to the cell growth chamber and the cell trap, and a second valve configured to selectively allow flow of fluid through the cell outlet.

12. The system of claim 1, further comprising a by-pass conduit configured for passage of fluid to the cell trap from a fluid reservoir.

13. The system of claim 1, further comprising a growth chamber inlet conduit configured for passage of fluid from a fluid reservoir to the growth chamber.

14. The system of claim 1, further comprising at least one pump for pumping fluid through the fluid flow system.

15. The system of claim 14, wherein the at least one pump is not positioned between the cell growth chamber and the cell trap.

16. The system of claim 1, further comprising a collection container fluidly coupled to the cell trap for collecting harvested cells from the cell trap.

17. The system of claim 16, wherein the collection container is detachable from the system.

18. The system of claim 1, wherein the cell growth chamber comprises an inlet, an outlet, and one or a plurality of cell culture plates positioned between the inlet and the outlet.

19. The system of claim 18, wherein the plurality of cell culture plates are arranged in a stacked configuration defining a plurality of flow channels through the cell growth chamber, and are oriented such that, in use, cells detached from the cell culture plates travel along the flow channels and through the outlet.

20. The system of claim 1, further comprising at least one sensor configured to monitor at least one fluid property of fluid in the system.

21. The system of claim 1, further comprising a sensor configured to monitor the volume of cells within the cell trap.

22. The system of claim 1, further comprising a controller configured to automatically control the fluid flow system to switch between the first mode of operation and the second mode of operation.

23. The system of claim 22, wherein the controller is configured to automatically switch the fluid flow system to the second mode of operation when a quantity of cells collected in the cell trap reaches a predetermined threshold.

24. (canceled)

25. A method for continuous bioprocessing of adherent cells, the method comprising:
selectively switching a fluid flow system between a first mode of operation and a second mode of operation,
wherein the first mode of operation comprises directing fluid through a cell growth chamber towards a cell trap and collecting cells from the cell growth chamber in the cell trap,
wherein the second mode of operation comprises directing fluid to the cell trap and through a cell trap outlet to thereby harvest cells collected in the cell trap.

26. The method of claim 25, wherein in the first mode of operation, fluid exiting the cell trap is recirculated through the cell growth chamber.

27. The method of claim 25, wherein in the first mode of operation, at least a portion of fluid exiting the cell trap is selectively directed towards a waste circuit and removed from the fluid flow system.
